(19) United States
(12) Patent Application Publication
(10) Pub. No.: US 2019/0358623 A1
(43) Pub. Date: Nov. 28, 2019

(54) A SYSTEM FOR MICRODROPLET MANIPULATION

(71) Applicant: SHILPS SCIECES PRIVATE LIMITED, Karnataka Bangalore (IN)

(72) Inventors: Ashwin LAL, Karnataka Bangalore (IN); Ichcha MANIPUR, Karnataka Bangalore (IN)

(21) Appl. No.: 16/484,135

(22) PCT Filed: Feb. 7, 2018
(86) PCT No.: PCT/IN2018/050064

§ 371 Date: Aug. 7, 2019

(30) Foreign Application Priority Data
Feb. 7, 2017 (IN) 201741004451

(51) Int. Cl. B01L 3/02; B01L 3/00; G01N 35/02; G01N 35/10; G01Q 80/00

(52) U.S. Cl. CPC B01L 3/022; G01N 2035/1034; G01N 35/028; G01N 35/10; G01Q 80/00; B01J 2219/00317; B01J 2219/0065; B01J 2219/00691; B01L 2200/0605; B01L 2200/0621; B01L 2200/0642; B01L 2200/0668; B01L 2300/0829; B01L 2400/027; B01L 2400/0421; B01L 2400/0427; B01L 2400/0487; B01L 3/5085

(57) ABSTRACT

The invention provides a method for manipulation of microdroplets in a microdroplet array. The method includes selecting at least one microdroplet from the microdroplet array and identifying at least one object trapped within the selected microdroplet. Subsequent to identifying the manipulation, a multi-functional probe specific to the determined manipulation is selected. The object identified is then subjected to manipulation. The invention further provides a system for manipulation of microdroplets in a microdroplet array.

### FIELD OF INVENTION

The invention generally relates to the field of microfluidics and particularly to a system for microdroplet manipulation.

### BACKGROUND

Microfluidic arrays generally include fluid or droplets, in the range of microliters (10⁻⁶) to picoliters (10⁻¹²). Each droplet in the array includes at least one object of interest. Examples of object of interest includes but is not limited to cells, hydro gels, DNA, RNA, enzymes, tags or other materials or fluids of interest within them. Hence, it is important to analyze and manipulate the droplets of a given microfluidic array.

There are various known methods for droplet manipulation. One such method is disclosed in the PCT application No. WO2015 061462A1 which describes an exporting device. The exporting device includes a pipette, a tube, a hollow needle and other such devices. The exporting device is used for exporting micro-objects from a microfluidic device, or within another location inside the microfluidic device. The export device aids in transporting small volumes of fluid media. However, one significant disadvantage of the system is that the system works with continuous fluid media and hence does not achieve true isolation.

Another method of spatially manipulating micro objects, such as single cells with the help of an AFM probe, is disclosed in US patent application US20130105034. This probe is used in manipulation of objects such as bacteria, biological cells, neurons, or submicron objects such as virus or nanoparticle. However, the AFM probe disclosed facilitates limited manipulation and is designed specifically for sub picoliter manipulations.

Another method of detection of analytes on a substrate using nanopositioners coupled to a nanospray mass spectrometry (NMS) system is discussed in US patent application US20120085900. The nanopositioner system utilizes one or more probes, grippers, capillary tips and any other suitable accessory for extraction of analytes with the help of pressure injector when placed in close proximity with the substrate to be analyzed. The analysis/identification of the analytes is done with the help of a mass spectrometer. The patent describes that this system can also be coupled to a microfluidic system. However, the nanomanipulator tip facilitates for only transfer of sample from one platform to another and does not achieve manipulation of sample of interest.

Hence, there is a need for a system that facilitates manipulation of droplets that has ease of operation using a probe for picoliter to nanoliter range in a microfluidic system/array and can implement various workflows of cell line engineering or other assays on the array using the same probe.

### BRIEF DESCRIPTION OF DRAWINGS

So that the manner in which the recited features of the invention can be understood in detail, some of the embodiments are illustrated in the appended drawings. It is to be noted, however, that the appended drawings illustrate only typical embodiments of this invention and are therefore not to be considered limiting of its scope, for the invention may admit to other equally effective embodiments.

FIG. 1(a) shows a schematic representation of a system for microdroplet manipulation, according to an embodiment of the invention.

FIG. 1 (b) shows a schematic representation of a system for microdroplet manipulation, according to an alternate embodiment of the invention.

FIG. 2(a) shows one configuration of the tip, according to an embodiment of the invention.

FIG. 2(b) shows another configuration of the tip, according to an embodiment of the invention.

FIG. 2(c) shows yet another configuration of the tip, according to an embodiment of the invention.

FIG. 3 generally shows static images of the microdroplet array, according to an example of the invention.

FIG. 4 shows transport of a microdroplet using the probe, according to an example of the invention.

FIG. 5(a) shows an intact microdroplet, according to an example of the invention.

FIG. 5(b) shows partial retention of the microdroplet in the tip of the microdroplet, according to an example of the invention.

FIG. 5(c) shows splitting of the microdroplet, according to an example of the invention.

FIG. 6(a) shows two different microdroplets for merging, according to an example of the invention.

FIG. 6(b) shows merging of two microdroplets, according to an example of the invention.

FIG. 7(a) shows an intact microdroplet for mixing of contents within the cell, according to an example of the invention.

FIG. 7(b) shows mixing of the contents within the microdroplet, according to an example of the invention.

FIG. 8(a) shows a genetic material outside the cell for electroporation, according to an example of the invention.

FIG. 8(b) shows electroporation of the genetic material inside the cell, according to an example of the invention.

FIG. 9(a) shows an intact cell within the microdroplet, according to an example of the invention.

FIG. 9(b) shows lysis of cell within the microdroplet, according to an example of the invention.

### SUMMARY OF THE INVENTION

One aspect of the invention provides a method for manipulation of microdroplets in a microdroplet array. The method includes selecting at least one microdroplet from the microdroplet array, identifying at least one object trapped within the selected microdroplet and determining at least one manipulation specific to the droplet along with the identified object. Subsequent to identifying the manipulation, a multi-functional probe specific to the determined manipulation is selected. The object identified is then subjected to manipulation.

Another aspect of the invention provides a system for manipulation of microdroplets in a microdroplet array.

### BRIEF DESCRIPTION OF THE INVENTION

Various embodiments of the invention provide a method for manipulation of microdroplet in pico to nano liter range in a microdroplet array. The method for manipulation includes selecting a target microdroplet. Subsequent to the selection of the microdroplet, the microdroplet is subjected to a manipulation. Examples of manipulation include but are not limited to sensing, splitting, merging, mixing, electroporation, electro-coalescence and transfer in a microdroplet array. The method described hereinabove briefly shall be explained in detail.

The step of selection is achieved by identifying at least one object within a given microdroplet in a well of a microdroplet array. The identification of the object within the microdroplet is achieved using image processing techniques. Examples of image processing techniques include but are not limited to image segmentation, feature extraction, pattern recognition, image enhancement and machine learning approaches. In one example, the identification of the object is achieved by initially obtaining a static image of each of the microdroplet in the microdroplet array and subsequently applying the decision making algorithm to the obtained static image. Various parameters are obtained from the algorithm. The obtained parameters are then stored for retrieving the same during manipulation.

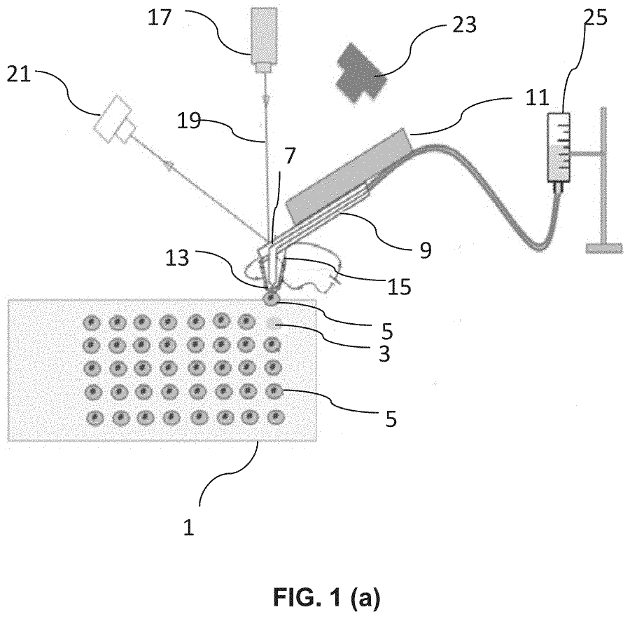

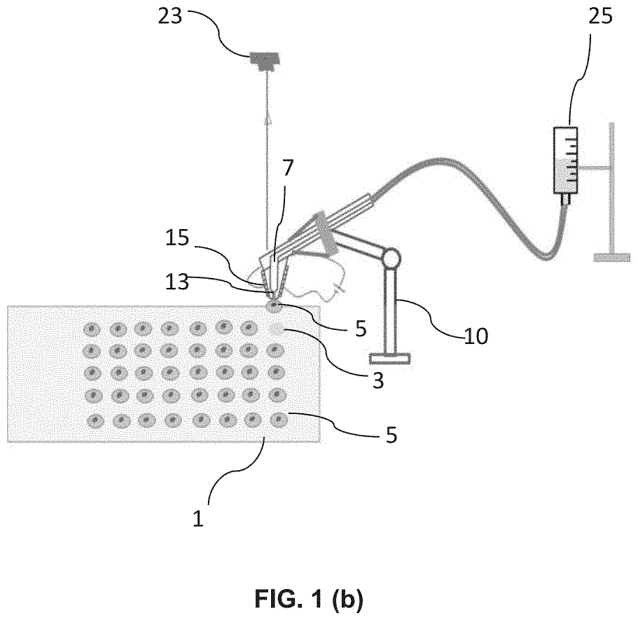

The invention further provides a system for manipulation of microdroplets in a microdroplet array using a multifunctional probe. The system for micro droplet manipulation includes a microdroplet array. The multi-functional probe is operably coupled to the micro droplet array. An identification module is configured for initially selecting a micro droplet within the array and subsequently identifying an object within the selected micro droplet. The identification module is operably coupled the micro droplet array. A manipulation module is operably coupled to the multi functional probe, wherein the manipulation module is configured to receive inputs from the identification module. Each of the aforementioned, shall be explained in detail herein below. FIG. 1(a) shows a schematic representation of a system for microdroplet manipulation, according to an embodiment of the invention. The system comprises of a microdroplet array 1. The microdroplet array consists of a series of wells 3. The wells may be 10-1000 m in depth and diameter. Each well contains a microdroplet 5. The microdroplet may be a water in oil emulsion droplet, an oil in water emulsion droplet or a droplet of a single fluid. Typically, one microdroplet occupies one micro well. The microdroplets contain objects including but not limited to cells, hydro gels, DNA, RNA, enzymes, tags or other materials or fluids of interest within them. There may be a layer of oil on the surface of the microdroplet array 1.

The microdroplet 5 can be generated using a microfluidic device and dispensed on to the microdroplet array 1. In one example of the invention, the microdroplet array 1 is made up of a material which includes but is not limited to a glass substrate, an acrylic, a PDMS, a COC, a metal and a silicon. The wells of the microdroplet array 1 are made using femto second laser machining. The microdroplet array 1 is provided with a probe 7. The multi-functional probe 7 includes a replaceable tip 13. Each tip is specific for a manipulation. A plurality of electrodes 15 is positioned proximal to the tip 13. The probe 7 can have multiple configurations. In one embodiment of the invention, the probe 7 is housed in a cantilever 9 supported by a cantilever beam 11. The probe 7 includes a tip 13. The tip 13 of the probe 7 can have multiple configurations. The probe 7 is also provided with electrodes 15. The electrodes 15 are positioned proximal to the tip 13 of the probe 7. In one example of the invention, the electrodes 15 are used for sensing the presence of objects in the microdroplet 5.

An identification module is configured for initially selecting a micro droplet within the array and subsequently identifying an object within the selected micro droplet. The identification module is operably coupled the micro droplet array. the identification module comprises of a vision guided system for capturing static image of the micro droplet array. An analyzer engine is coupled to the vision guided device for analyzing the static images obtained. A communication unit is coupled to the analyzer for delivering instructions to the manipulation module. The identification module includes a vision guided system 23. The vision guided system 23 is positioned over the microdroplet array 1. The vision guided system 23 is configured to perform multiple functions. A primary function of the vision guided system 23 is to take static images of the microdroplet array 1. A secondary function is to facilitate analysis of the images obtained. A tertiary function of the system is to send instructions to the probe 7 for performing manipulation of the target microdroplet of the microdroplet array 1. The vision guided system 23 includes an image capturing device, an analyser coupled to the image capturing device and a storage unit.

The manipulation module includes an automated positioning arrangement configured for receiving input from the identification module and positioning the multi-functional probe 7 over a desired micro droplet. The manipulation module also includes a flow control device 25 that is configured for regulating the operation of the multi-functional probe 7. In one example of the invention, the automated positioning arrangement includes a laser source 17 that is operably coupled to the probe 7. The laser source 17 generates a laser beam 19 of a defined wavelength. A detector 21 is coupled to the laser source 17 for measuring the deflection of the cantilever 9. The detector 21 measures the force between the tip 13 and the microdroplet surface 5. The laser beam 19 along with the detector 21 allows positioning of the probe 7. A flow control device 25 is operably coupled to the probe 7. The flow control device 25 includes but is not limited to a pressure pump, a gravity based pressure control systems and any other such flow control devices. The flow control device 25 is configured for regulating the operation of the probe 7. Examples of manipulation include but is not limited to sensing, splitting, merging, mixing, electro-coalescence, electroporation, transfer and/or combinations thereof, of the droplet along with the identified object.

FIG. 1 (b) shows a schematic representation of a system for microdroplet manipulation, according to an alternate embodiment of the invention. The system comprises of a microdroplet array 1. The microdroplet array consists of a series of wells 3. The wells may be 10-1000 m in depth and diameter. Each well contains a microdroplet 5. The microdroplet may be a water in oil emulsion droplet, an oil in water emulsion droplet or a droplet of a single fluid. Typically, one microdroplet occupies one micro well. The microdroplets contain objects including but not limited to cells, hydro gels, DNA, RNA, enzymes, tags or other materials or fluids of interest within them. There may be a layer of oil on the surface of the microdroplet array 1.

The microdroplet 5 can be generated using a microfluidic device and dispensed on to the microdroplet array 1. In one example of the invention, the microdroplet array 1 is made up of a material which includes but is not limited to a glass substrate, an acrylic, a PDMS, a COC, a metal and a silicon. The wells of the microdroplet array 1 are made using femto second laser machining.

The microdroplet array 1 is provided with a probe 7. The probe 7 can have multiple configurations. The probe 7 includes a tip 13. The tip 13 of the probe 7 can have multiple configurations. The probe 7 is also provided with electrodes 15. The electrodes 15 are positioned proximal to the tip 13 of the probe 7. In one example of the invention, the electrodes 15 are used for sensing the presence of objects in the microdroplet 5.

An identification module is configured for initially selecting a micro droplet within the array and subsequently identifying an object within the selected micro droplet. The identification module is operably coupled the micro droplet array. the identification module comprises of a vision guided system for capturing static image of the micro droplet array. An analyzer engine is coupled to the vision guided device for analyzing the static images obtained. A communication unit is coupled to the analyzer for delivering instructions to the manipulation module. The identification module includes a vision guided system 23. The vision guided system 23 is positioned over the microdroplet array 1. The vision guided system 23 is configured to perform multiple functions. A primary function of the vision guided system 23 is to take static images of the microdroplet array 1. A secondary function is to facilitate analysis of the images obtained. A tertiary function of the system is to send instructions to the probe 7 for performing manipulation of the target microdroplet of the microdroplet array 1. The vision guided system 23 includes an image capturing device, an analyser coupled to the image capturing device and a storage unit.

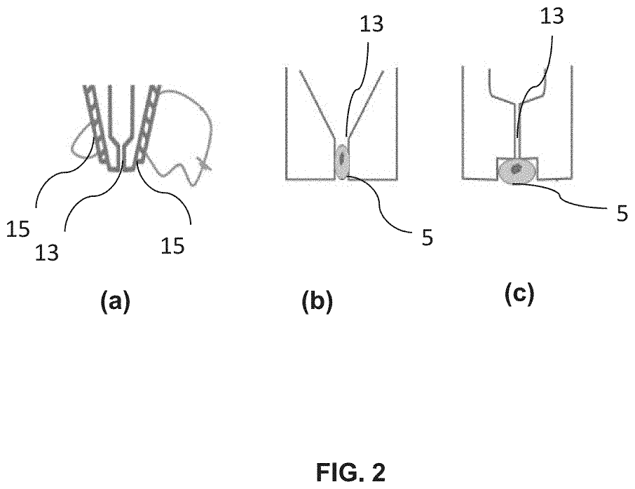

The manipulation module includes an automated positioning arrangement configured for receiving input from the identification module and positioning the multi-functional probe 7 over a desired micro droplet. The manipulation module also includes a flow control device 25 that is configured for regulating the operation of the multi-functional probe 7. In another example of the invention, the automated positioning arrangement includes an XY positioning system 10. The flow control device 25 is operably coupled to the probe 7. The flow control device 25 includes but is not limited to a pressure pump, a gravity based pressure control systems and any other such flow control devices. The flow control device 25 is configured for regulating the operation of the probe 7. Examples of operation include but are not limited to holding, splitting and merging of microdroplets. FIG. 2 generally shows different configurations of the tip 13 of the probe 7, according to an embodiment of the invention.

FIG. 2(a) shows one configuration of the tip, according to an embodiment of the invention. The probe 7 is provided with electrodes 15 in proximity to the tip 13. The electrodes 15 proximal to the tip 13 are used for identifying the presence of objects in the microdroplet. The electrodes in the tip of the probe use high frequency impedance measurements to detect the objects of interest within the microdroplets or detect the presence or absence of the microdroplet itself.

FIG. 2(b) shows another configuration of the tip, according to an embodiment of the invention. The microdroplet 5 is held within the tip 13.

FIG. 2(c) shows yet another configuration of the tip, according to an embodiment of the invention. The microdroplet 5 is held at the tip 13.

The method and system as described herein above is utilized for manipulation of the target microdroplet. The manipulation achieved shall be explained herein below as examples of the invention.

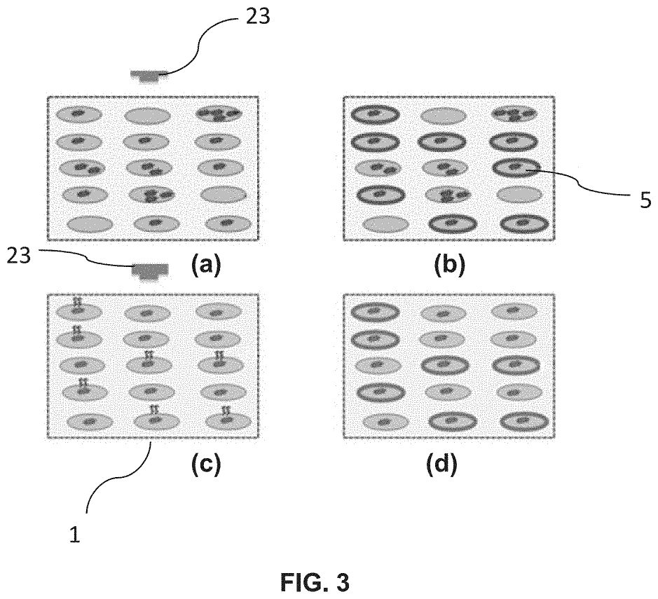

FIGS. 3(a) and 3(b) generally shows static images of the microdroplet array for identification of target droplet, according to one example of the invention. All the wells in the microdroplet array 1 are imaged with a vision guided system 23 and pre-processed with various decision making algorithms. The algorithms help to designate coordinates to the wells in the microdroplet array. The identified coordinates are then used to position a probe 7 over the well.

FIGS. 3(c) and 3(d) generally shows static images of the microdroplet array for identification of target droplet, according to another example of the invention. The object inside the droplets is tagged using a fluorescence emitter. The identification of target droplet is achieved with the help of fluorescence emission from cells. The probe 7 is then positioned over the identified target droplet.

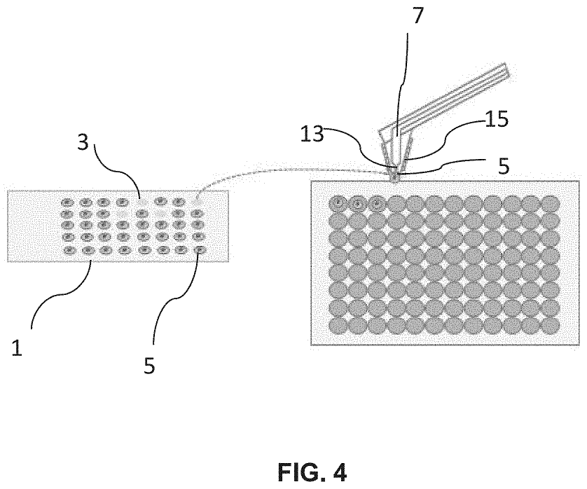

FIG. 4 shows transport of the microdroplet 5 using the probe 7, according to an example of the invention. The transport of the microdroplet 5 is achieved using either a flow control device or dielectrophoresis. The flow control device 25 (not shown) includes but is not limited to a pressure pump, a gravity based pressure control systems and any other such flow control devices. In an alternate example of the invention, dielectrophoresis can be used to pull the microdroplet 5 to the tip 13, with AC voltage applied at 1-100 kHz and between tens to hundreds of volts.

In another example of the invention, the tip 13 is brought in contact with the microdroplet 5 and a DC voltage of tens to hundreds of volts is applied at the electrode 15, which leads to electrowetting making the microdroplet 5 stick to the tip 13.

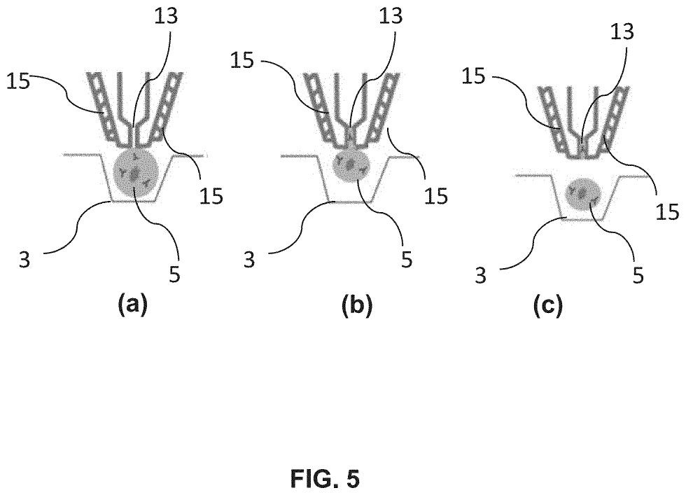

FIG. 5 generally shows splitting of a microdroplet 5, according to another example of the invention.

FIG. 5(a) shows an intact microdroplet, according to an example of the invention. The tip 13 is brought in close contact with the microdroplet 5 in the well 3. The microdroplet 5 is made to adhere to the tip 13, using either a flow control device or dielectrophoresis. The flow control device 25 (not shown) includes but is not limited to a pressure pump, a gravity based pressure control systems and any other such flow control devices. Once the tip 13 is sufficiently close to the microdroplet 5, pressure is applied using a flow control device 25 and the microdroplet 5 is held at the tip 13. In an alternate example of the invention, dielectrophoresis can be used to pull a microdroplet 5 to the tip 13, with AC voltage applied at 1-100 kHz and between tens to hundreds of volts.

In another example of the invention, the tip 13 is brought in contact with the microdroplet 5 and a DC voltage of tens to hundreds of volts is applied at the electrode 15, which leads to electrowetting making the microdroplet 5 stick to the tip 13.

FIG. 5(b) shows partial retention of the microdroplet in the tip 13, according to an example of the invention. The opening of the tip 13 is about 10-50% of the microdroplet size. When controlled negative pressure is applied at the tip, the microdroplet deforms and a part of the microdroplet is partially retained in the tip.

FIG. 5(c) shows splitting of the microdroplet, according to an example of the invention. The tip 13 is retracted leading to a split in the microdroplet and one part of the microdroplet is transferred.

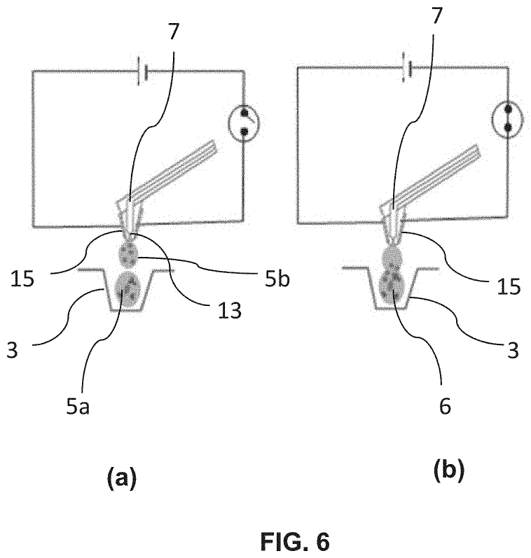

FIG. 6(a) shows two different microdroplets for merging, according to an example of the invention. The tip 13 having a second microdroplet 5b is brought in close contact with the well 3 having the first microdroplet 5a.

FIG. 6(b) shows merging of two microdroplets, according to an example of the invention. When sufficient voltage is applied to the electrode 15, the microdroplets can be coalesced to form a new droplet 6.

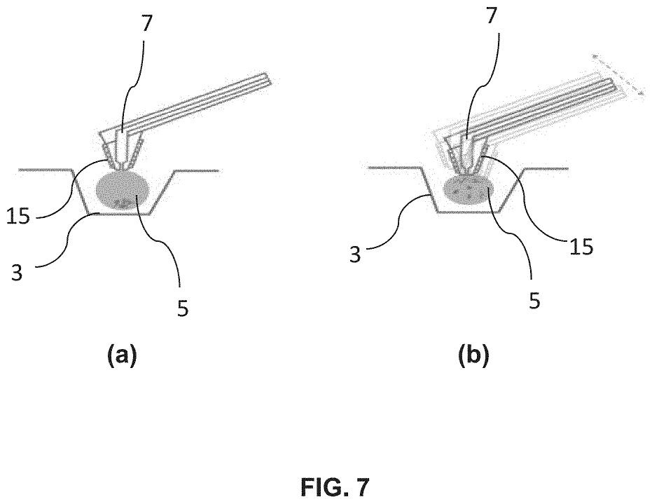

FIG. 7(a) shows an intact microdroplet for mixing of contents within the cell, according to an example of the invention. The tip 13 is brought in close contact with the microdroplet 5 in the well 3. The microdroplet 5 is made to adhere to the tip 13, using either a flow control device or dielectrophoresis. The flow control device 25 (not shown) includes but is not limited to a pressure pump, a gravity based pressure control systems and any other such flow control devices. In an alternate example of the invention, dielectrophoresis can be used to pull a microdroplet 5 to the tip 13, with AC voltage applied at 1-100 kHz and between tens to hundreds of volts.

In another example of the invention, the tip 13 is brought in contact with the microdroplet 5 and a DC voltage of tens to hundreds of volts is applied at the electrodes, which leads to electrowetting making the microdroplet 5 stick to the tip 13.

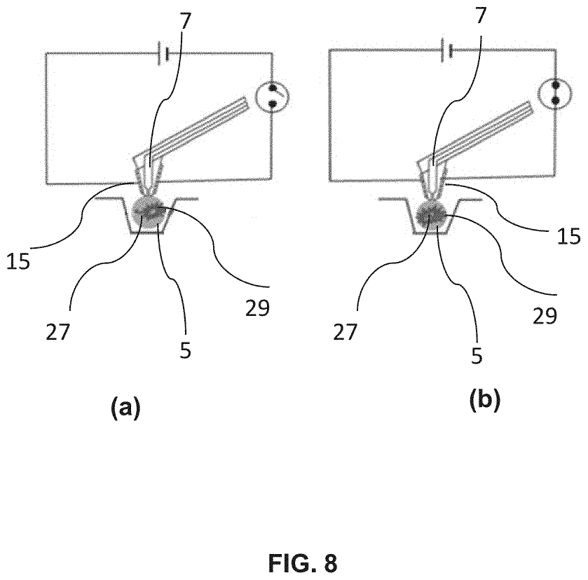

FIG. 7(b) shows mixing of the contents within the microdroplet 5, according to an example of the invention. The probe 7 is used to mix the contents within the microdroplets to distribute the contents uniformly. The probe 7 is vibrated at a few Hz to a hundred kHz with the help of a positioner (not shown). The positioner could be a combination of motor and piezo elements. FIG. 8 generally shows electroporation of contents inside the microdroplet using the probe 7, according to an example of the invention.

FIG. 8(a) shows a genetic material 27 outside a cell 29 for electroporation, according to an example of the invention. The probe 7 is used to cause electroporation by applying a voltage to the microdroplet 5. When the voltage is applied to the electrodes on the probe 7, the genetic material 27 which is outside the cell 29 moves inside the cell, as shown in FIG. 8(b).

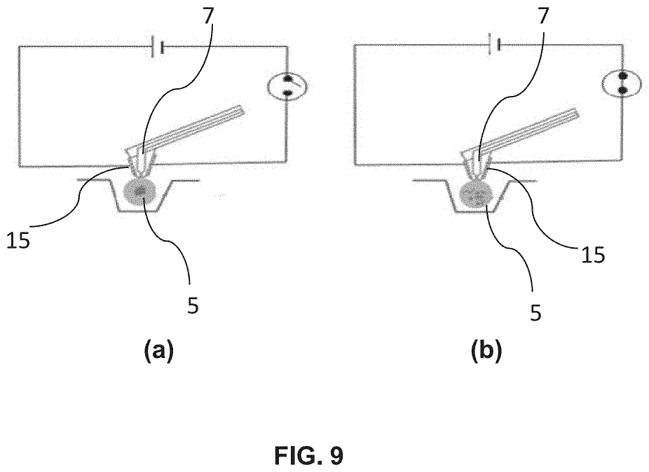

FIG. 9 generally shows lysis of cells inside the microdroplet 5 using the probe 7, according to an example of the invention.

FIG. 9(a) shows an intact cell within the microdroplet 5, according to an example of the invention. When sufficient voltage is applied to the electrode 15, the cell encapsulated within the microdroplet 5 undergoes lysis, as shown in FIG. 9(b). The system allows high throughput analysis of microdroplet array through a plurality of probes connected together. The probe is capable of sensing the contents of a cell using a decision based system. A single probe is capable of carrying out multiple functions. The system allows adoption of probe in a workflow for cell line engineering, sequencing preparation and other assays. The system can further have plurality of connected probes capable of carrying out either identical and/or multiple functions in a workflow for cell line engineering, sequencing preparation and other assays. The system provides ease of configuration and adoption with any microfluidic device capable of generating micro droplets.

The foregoing description of the invention has been set for merely to illustrate the invention and is not intended to be limiting. Since modifications of the disclosed embodiments incorporating the spirit and substance of the invention may occur to person skilled in the art, the invention should be construed to include everything within the scope of the appended claims and equivalents thereof.

We claim:

1. An automated method for manipulation of a micro droplet array, the method comprising:
selecting at least one microdroplet from the microdroplet array;
identifying at least one object trapped within the selected microdroplet;
determining at least one manipulation specific to the droplet along with the identified object;
selecting at least one multi-functional probe specific to the determined manipulation; and
performing the said manipulation of the droplet along with the identified object with the selected multi-functional probe.

2. The method of claim 1, wherein the object comprises of a single cell, a hydro gel, a DNA, a RNA, an enzyme, a compound, a polypeptide, a polynucleotide, a dendrimer, a polymer and/or combinations thereof.

3. The method of claim 1, wherein the identification of the droplet is achieved by initially obtaining a static image of each of the microdroplet in the microdroplet array and subsequently applying at least one decision making algorithm to the obtained static image to obtain the position of the object within the microdroplet.

4. The method of claim 1, wherein the manipulation comprises of sensing, splitting, merging, mixing, electro-coalescence, electroporation, transfer and/or combinations thereof of the droplet along with the identified object.

5. A system for micro droplet manipulation, the system comprises of:
a microdroplet array 1;
a multi-functional probe 7 operably coupled to the micro droplet array 1;
an identification module configured for initially selecting a micro droplet 5 within the micro droplet array 1 and subsequently identifying an object within the selected micro droplet 5, wherein the identification module is operably coupled the micro droplet array; and
a manipulation module operably coupled to the multi functional probe 7, wherein the manipulation module is configured to receive inputs from the identification module.

6. The system of claim 5, wherein the micro droplet array can be obtained by a microfluidic device.

7. The system of claim 5, wherein the multi-functional probe 7 comprises of:
a replaceable tip 13, wherein each tip is specific for a manipulation; and
a plurality of electrodes 15 positioned proximal to the tip.

8. The system of claim 5, wherein the identification module comprises of:
a vision guided system 23 for capturing static image of the micro droplet array;
an analyzer engine coupled to the vision guided device for analyzing the static images obtained; and
a communication unit coupled to the analyzer for delivering instructions to the manipulation module.

9. The system of claim 5, wherein the manipulation module comprises of:
an automated positioning arrangement configured for receiving input from the identification module and positioning the multi-functional probe 7 over a desired micro droplet;
a flow control device 25 configured for regulating the operation of the multi-functional probe 7.

10. The system of claim 5 wherein the manipulation comprises of comprises of sensing, splitting, merging, mixing, electro-coalescence, electroporation, transfer and/or combinations thereof, of the droplet along with the identified object.
